(19) United States
(12) Patent Application Publication
(10) Pub. No.: US 2022/0331376 A1
(43) Pub. Date: Oct. 20, 2022

(54) MIXOTROPHIC PROBIOTIC COMPOSITIONS AND USES THEREOF IN THE TREATMENT OF BLOATING

(71) Applicant: WHITE DOG LABS, New Castle, DE (US)

(72) Inventors: Alon KARPOL, Tel Mond (IL); Shawn William JONES, Bear, DE (US); Bryan P. TRACY, Wilmington, DE (US); Aharon M. EYAL, Jerusalem (IL)

(21) Appl. No.: 17/639,931

(22) PCT Filed: Sep. 4, 2020
(86) PCT No.: PCT/IB2020/058275

§ 371 Date: Mar. 3, 2022

Related U.S. Application Data
(60) Provisional application No. 62/896,003, filed on Sep. 5, 2019.

(51) Int. Cl. A61K 35/741; A61K 35/742; A61K 35/747; A61K 36/064; A61P 1/14; A61K 35/744; A61K 35/745

(52) U.S. Cl. CPC A61K 35/741; A61K 35/742; A61K 35/747; A61K 36/064; A61P 1/14; A61K 35/744; A61K 35/745; A61K 2035/115

(57) ABSTRACT

Provided is a probiotic composition, comprising at least one genus of mixotrophic microorganism and a carrier and pharmaceutical preparations comprising such a composition for use in the treatment of bloating in an animal. Also provided are methods of use of such pharmaceutical preparations in the treatment of bloating in an animal.

### CROSS-REFERENCE TO RELATED APPLICATION

The present application gains priority from U.S. Provisional Application No. 62/896,003 filed 5 Sep. 2019, which is incorporated by reference as if fully set-forth herein.

### FIELD OF THE INVENTION

The present invention, in at least some embodiments, relates to probiotic compositions and in particular to probiotic compositions comprising at least one genus of mixotrophic microorganism and a carrier for use in the treatment of bloating.

### BACKGROUND OF THE INVENTION

Bloating is a condition in which the belly feels full and tight, often due to gas accumulation in the gastrointestinal tracks (GIT). While for most healthy people, bloating is a transient, inconvenient condition, which can cause only a short temporary discomfort, for patients with Inflammatory Bowel Diseases (IBD), such as Crohn's Disease (CD) and Ulcerative Colitis (UC), bloating can cause gas sensitivity which leads to pain, cramping and diarrhea.

Currently there are several treatments for bloating such as Antibiotics, Prokinetics, Antispasmodics, Opioid Agents, Antidepressants and Stimulants of Fluid Secretion. Some of those treatments have serious side effects while others have limited effect on bloating. Hence there is a need for a more effective, safer product which can be used across a spectrum of conditions: from healthy people with transient bloating condition to lactose intolerance patients to IBD patients with chronic gas bloating.

### SUMMARY OF THE INVENTION

According to an aspect of the present invention, there is provides a probiotic composition, comprising at least one genus of mixotrophic microorganism and a carrier for use in the treatment of bloating.

According to an embodiment, at least one genus of mixotrophic microorganism comprises at least two, at least three or at least four genera of mixotrophic microorganisms.

According to an embodiment, the carrier is configured to maintain an anaerobic environment for said microorganism.

According to an embodiment, the microorganism has at least one property selected from the group consisting of butanoate metabolism, obligate anaerobic growth, gas fixation via the reductive acetyl-coenzyme A pathway, tolerance to bile salts at concentration greater than 0.05%, tolerance to pH of less than 4.5, spore formation, lactose utilization, and self-aggregation.

According to an embodiment, the at least one genus is selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Enterococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

According to an embodiment, the microorganism is present as a live vegetative culture.

According to an embodiment, the microorganism is present as a sporulated culture i.e. a culture in which an average of at least 0.1% of the cells are in spores-state.

According to an embodiment, the carrier is selected from the group consisting of oxygen reducer (such as at least one selected from the group consisting of cysteine hydrochloride, sodium sulfide, sodium sulfite, sodium metabisulfite and combinations thereof), cellulose, and carbohydrates.

According to an embodiment, the composition further comprises microorganisms of at least one species selected from the group consisting of Bacillus amyloliquefaciens; Bacillus timonensis; Bacillus coagulans; Bacillus licheniformis; Bacillus megaterium; Bacillus mesentricus; Bacillus polymyxa; Bacillus subtilis; Bifidobacterium animalis; Bifidobacterium bifidium; Bifidobacterium bifidus; Bifidobacterium thermophilus; Bifidobacterium longum; Bifidobacterium pseudolongum; Bifidobacterium lactis; Clostridium butyricum; Enterococcus faecium; Enterococcus faecalis; Escherichia coli; Lactobacillus thermophilus; Lactobacillus acidophilus; Lactobacillus brevis; Lactobacillus bulgaricus; Lactobacillus casei; Lactobacillus delbrueckii; Lactobacillus subspecies; Lactobacillus bulgaricus; Lactobacillus farciminis; Lactobacillus fermentum; Lactobacillus gallinarum; Lactobacillus jensenii; Lactobacillus paracasei; Lactobacillus plantarum; Lactobacillus reuteri; Lactobacillus rhamnosus; Lactobacillus lactis; Lactobacillus salivarius; Lactobacillus sobrius; Megasphaera elsdenii; Pediococcus cidolactici; Propionibacterium shermanii; Propionibacterium freudenreichii; Propionibacterium acidipropionici; Propionibacterium jensenii; Saccharomyces boulardii; Saccharomyces cerevisiae; Saccharomyces servisia; Streptococcus faecalis; Streptococcus faecium; Streptococcus gallolyticus; Streptococcus salivarius; Streptococcus subsp.; Streptococcus thermophilus; Streptococcus bovis and combinations thereof.

According to an embodiment, the composition comprises a mixture of at least two different species of mixotrophic microorganisms. According to some such embodiments, the at least two different species show syntrophic behavior in an animal. According to some such embodiments, the syntrophic behavior is beneficial to the animal i.e. can prevent or reduce a condition or symptom thereof in the animal or provide a beneficial property such as a resistance to a detrimental condition.

According to an embodiment, the mixture of at least two different species comprises at least one microorganism selected from the group consisting of a CO₂-utilizing microorganism; a CO-utilizing microorganism; and an H₂-utilizing microorganism. According to some such embodiments, at least one of the CO₂-utilizing microorganism; the CO-utilizing microorganism; and the H₂-utilizing organism is an acetogen.

According to an embodiment, the mixture of at least two different species comprises at least one non-CO₂ utilizing microorganism.

According to an embodiment, the mixture of at least two different species comprises at least one acetate forming microorganism and at least one acetate-utilizing microorganism.

According to an embodiment, the mixture of at least two different species comprises at least one lactate forming microorganism and at least one lactate-utilizing organism.

According to an aspect of some embodiments of the present invention, there is provided a pharmaceutical preparation comprising the composition as disclosed herein for use in the treatment of bloating.

According to an embodiment, the pharmaceutical preparation comprises between 10² and 10¹⁰ colony forming units of the microorganism.

According to an embodiment, the pharmaceutical preparation comprises at least 10¹ Colony-Forming Units per milliliter of the microorganism.

According to an embodiment, the pharmaceutical preparation further comprises at least one selected from the group consisting of water, macronutrients, prebiotics, and probiotics and combinations thereof.

According to an aspect of some embodiments of the present invention, there is provided a method of treating bloating in an animal comprising administering to the animal an effective dose of a composition as disclosed herein, thereby improving the health of the animal.

According to an aspect of some embodiments of the present invention, there is provided a pharmaceutical preparation as disclosed herein for use in treating bloating in an animal.

According to an embodiment, the animal is selected from the group consisting of human, dog, cat, sheep, horse, chicken, swine, cattle, reptiles, goat, duck, turkey, fish, shrimp and crabs.

According to an embodiment, the microorganism colonizes at least a portion of a section of the digestive tract of the animal i.e. is present on a surface of the section of the digestive tract without causing disease in the anima.

According to some such embodiments, the section of the digestive tract is selected from the group consisting of duodenum, jejunum, ileum, small intestine, cecum and colon.

According to an embodiment, the effective dose is administered at least twice.

According to an embodiment, the effective dose is configured to provide at least 10 organism Colony-Forming Units per gram wet feces of the animal by a second day after administering the composition.

According to an embodiment, the colonizing is configured to provide at least 100 Colony-Forming Units per gram wet feces of the animal by a twentieth day after administering the composition.

According to an embodiment, the bloating is caused by at least one selected from the group consisting of an immune condition; a digestive condition; a pathogenic microorganism (including bacteria, viruses, fungi, protozoa); a disease of the gastrointestinal system (such as Irritable Bowel Syndrome (IBS), Celiac Disease or Inflammatory Bowel Disease, including Ulcerative Colitis, Crohn's Disease, Microscopic Colitis and Pauchitis or combinations thereof); an iatrogenic disturbance or dysbiosis caused by one selected from the group consisting of radiotherapy, chemotherapy, transplantation and combinations thereof; antibiotic-related diarrhea (such as related to treatment of Clostridium difficile-associated diarrhea or recurrent Clostridium difficile infections) and combinations thereof.

According to an embodiment, bloating cause abdominal cramp, such that treatment of bloating results in treatment of abdominal cramp.

According to an embodiment, the probiotic composition is configured to provide formation of butyric acid at a rate sufficient to reach butyric acid concentration of at least 0.1 millimolar in the digestive track of the animal.

According to an embodiment, the effective dose is configured to provide formation of butyric acid at a rate of at least 0.01 millimolar per hour in the digestive tract of the animal.

### DETAILED DESCRIPTION OF THE INVENTION

The present invention, in at least some embodiments thereof, provides mixotrophic probiotic compositions and uses thereof in the treatment of bloating.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As used herein, the term “mixotrophic microorganism” refers to a microorganism with the ability to fix inorganic feedstock (such as H₂, CO₂ or CO), and organic feedstock (such as glucose or xylose).

According to an embodiment, the inorganic feedstock is selected from the group consisting of H₂, CO₂ and CO or combinations thereof.

According to an embodiments, the organic feedstock is selected from the group consisting of glucose, xylose, glycogen, starch, cellulose, cellobiose, sucrose, maltose, lactose, mannose, galactose, fructose, inlulin, fructooligosaccharides, galactooligosaccharide, mannan oligosaccharide, ribose, Trehalose, dextrose, multidextrin, pectin and combinations thereof.

According to an embodiment, fixation of an inorganic feedstock can be accomplished through the reductive acetyl-coenzyme A pathway, also known as the Wood-Ljungdahl pathway. Alternatively or additionally, fixation of inorganic feedstock is accomplished by at least one carbon fixation pathway selected from the group consisting of the reductive pentose phosphate cycle; the reductive TCA cycle; the dicarboxylate-4-hydroxybutyrate Cycle; the 3-Hydroxypropionate-4-hydroxybutyrate cycle; the 3-Hydroxypropionate bicycle; the reductive acetyl-CoA pathway (acetogens); the reductive acetyl-CoA pathway (methanogens); the glycine synthase pathway (glycine reductase); the glycine synthase pathway (serine hydroxymethytransferase); the glyoxylate synthetase pathway and combinations thereof.

As used herein, the term “probiotic” refers to a live microorganism which provides health benefits to an animal when consumed, generally by restoring the balance of gut flora.

As used herein, the term “prebiotic” refers to a compound that induces the growth or activity of a probiotic.

As used herein, the term “bloating’ refers to sense of gassiness or a sense of being distended. According to some embodiments, functional bloating is determined by the Rome III diagnostic criteria: recurrent feeling of bloating or visible distention for at least 3 days per month; onset of symptoms at least 6 months prior to diagnosis; presence of symptoms for at least 3 months; and insufficient criteria to establish a diagnosis of irritable bowel syndrome, functional dyspepsia or other functional gastrointestinal disorder.

As used herein, the term “butanoate metabolism” (also known as “butyrate metabolism”) refers to the genes and pathways involving the formation of butyrate, the anion of butyric acid (butanoic acid), from acetyl-CoA. Genes can include, but are not limited to, thiolase (also known as acetyl-CoA acetyltransferase), 3-hydroxybutyl-CoA dehydrogenase, crotonase (also known as enoyl-CoA hydratase), butyryl-CoA dehydrogenase, trans-2-enoyl-CoA reductase, phosphate butyryltransferase, and butyrate kinase.

As used herein, the term “obligate anaerobic growth” refers to growth of a microorganism which is unable to survive in a composition having an oxygen composition of 100 microMolar or more.

As used herein, the term “gas fixation via the reductive acetyl-coenzyme A pathway” refers to a set of biochemical reactions wherein microorganisms use hydrogen as an electron donor and carbon dioxide as an electron acceptor and as a building block for biosynthesis.

As used herein, the term “tolerance to bile salts at a concentration greater than 0.05%” refers to survival of at least 1% of the cells exposed to the specified concentration of bile salts for a duration of at least 2 hours.

As used herein, the term “tolerance to pH of less than 4.5” refers to survival of at least 1% of the cells exposed to the specified pH for a duration of at least 2 hours.

As used herein, the term “spore formation” (also known as “sporulation”) refers to the formation of highly resistant, dormant structures having no metabolic activity, which are formed in some microorganisms in response to environmental conditions (such as depletion of nutrients) in order to help in survival of the microorganism.

As used herein, the term “lactose utilization” refers to the ability of a microorganism to uptake and catabolize lactose as a carbon source.

As used herein, the term “self-aggregation” (also known as “auto aggregation”) refers to the formation of multicellular clumps by bacteria of the same type.

As used herein, the term “anaerobic environment” refers to an environment having less than about 100 mM of oxygen.

As used herein, the term “live, vegetative culture” refers to culture in which a microorganism is able to grow and reproduce.

As used herein, the term “improving digestion” refers to improving a Protein Digestibility Corrected Amino Acid Score (PDCAAS) in an animal model by at least about 10%.

As used herein, the terms “CO₂-utilizing microorganism” and “CO-utilizing microorganism” refer to microorganisms having the ability to fix organic carbon dioxide and carbon monoxide, respectively, to form organic carbon compounds, while the terms “non-CO₂-utilizing microorganism” and “non-CO-utilizing microorganism” refer to microorganisms which are devoid of the aforementioned ability. As used herein, the term “syntrophic behavior” refers to a nutritional interdependence between two or more microorganisms. Such interdependence may be mutually beneficial but is not necessarily required for growth. According to an embodiment, the syntrophic behavior involves a first microorganism consuming a metabolite produced by a second microorganism, wherein the metabolite is harmful to the environment or to the second microorganism, resulting in the removal or reduction of the level of the harmful metabolite.

According to an embodiment, the syntrophic behavior occurs between one or more microorganisms within the mixture of microorganisms in the composition. Alternatively or additionally, the syntrophic behavior occurs between one or more microorganisms in the composition and one or more native microorganisms in the animal to which the composition is administered.

As used herein, the term “treating” includes preventing, curing, ameliorating, mitigating, and reducing the instances or severity of a condition or a symptom thereof.

As used herein, the term “effective dose” means the amount of an active substance that, when administered to a subject for treating a disease, disorder, or other undesirable medical condition, is sufficient to have a beneficial effect with respect to that disease, disorder, or condition. The effective dose will vary depending on the identity and formulation form of the active substance, the disease or condition and its severity, and the age, weight, and other relevant characteristics of the patient to be treated. Determining the effective amount of a given active substance is within the ordinary skill of the art and typically requires no more than routine experimentation.

According to an aspect of the present invention, there is provides a probiotic composition, comprising at least one genus of mixotrophic microorganism and a carrier for use in the treatment of bloating.

Without wishing to be bound to any one theory, the present inventors hypothesize that mixotrophic strains can treat bloating by one of the following mechanisms:

(i) consumption of gas generated by gas-producing microorganisms
(ii) competition for the sugars between the mixotrophic microorganism and the gas-producing organisms, thereby reducing the population of the gas-producing microorganisms. Consumption of sugars by the mixotrophic organism results in significantly less gas production, in some embodiments at least 20% less gas production.
(iii) mixotrophic consumption of both gases and sugars.

According to an embodiment, at least one genus of mixotrophic microorganism comprises at least two, at least three or at least four genera of mixotrophic microorganisms.

According to an embodiment, the carrier is configured to maintain an anaerobic environment for said microorganism.

According to an embodiment, the microorganism is characterized by having at least one characteristic selected from the group consisting of butanoate metabolism, obligate anaerobic growth, gas fixation via the reductive acetyl-coenzyme A pathway, tolerance to bile salts at concentration greater than 0.05%, tolerance to pH of less than 4.5, spore formation, lactose utilization, and self-aggregation.

According to an embodiment, the at least one genus is selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Enterococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

According to an embodiment, the composition comprises at least two, at least three, at least four at least five, at least six, at least seven, at least eight, at least nine or at least ten different genera of microorganisms.

According to an embodiment, the composition comprises at least at least two, at least three, at least four at least five, at least six, at least seven, at least eight, at least nine or at least ten different species of microorganisms. In some such embodiments, the species are of same genus. In some such embodiments, the species are of different genera.

According to an embodiment, the microorganism is present as a live vegetative culture.

According to an embodiment, the microorganism is present as a sporulated culture.

According to an embodiment, the carrier is selected from the group consisting of oxygen reducer, cellulose, and carbohydrates, such as cysteine hydrochloride, sodium sulfide, sodium sulfite, sodium metabisulfite and combinations thereof.

According to an embodiment, the composition further comprises microorganisms of at least one species selected from the group consisting of Bacillus amyloliquefaciens; Bacillus timonensis; Bacillus coagulans; Bacillus licheniformis; Bacillus megaterium; Bacillus mesentricus; Bacillus polymyxa; Bacillus subtilis; Bifidobacterium animalis; Bifidobacterium bifidum; Bifidobacterium bifidus; Bifidobacterium thermophilus; Bifidobacterium longum; Bifidobacterium pseudolongum; Bifidobacterium lactis; Clostridium butyricum; Enterococcus faecium; Enterococcus faecalis; Escherichia coli; Lactobacillus thermophilus; Lactobacillus acidophilus; Lactobacillus brevis; Lactobacillus bulgaricus; Lactobacillus casei; Lactobacillus delbrueckii; Lactobacillus subspecies; Lactobacillus bulgaricus; Lactobacillus farciminis; Lactobacillus fermentum; Lactobacillus gallinarum; Lactobacillus jensenii; Lactobacillus paracasei; Lactobacillus plantarum; Lactobacillus reuteri; Lactobacillus rhamnosus; Lactobacillus lactis; Lactobacillus salivarius; Lactobacillus sobrius; Megasphaera elsdenii; Pediococcus acidolactici; Propionibacterium shermanii; Propionibacterium freudenreichii; Propionibacterium acidipropionici; Propionibacterium jensenii; Saccharomyces boulardii; Saccharomyces cerevisiae; Saccharomyces servisia; Streptococcus faecalis; Streptococcus faecium; Streptococcus gallolyticus; Streptococcus salivarius; Streptococcus subsp.; Streptococcus thermophilus; Streptococcus bovis and combinations thereof.

According to an embodiment, the composition comprises a mixture of at least two different species of mixotrophic microorganisms. In some such embodiments, the composition comprises at least two, at least three, at least four, at least five, at least six, at least seven, at least eight, at least nine or at least ten different species of microorganisms.

According to some such embodiments, the at least two different species show syntrophic behavior in an animal. According to some such embodiments, the syntrophic behavior is beneficial to the animal.

According to an embodiment, the mixture of at least two different species comprises at least one microorganism selected from the group consisting of a CO₂-utilizing microorganism (such as Eubacterium aggregans, Blautia producta and combinations thereof); a CO-utilizing microorganism (such as Clostridium ljungdahlii, Clostridium autoethanogenum, Clostridium scatologenes Clostridium drakei Clostridium carboxidivorans Butyribacterium, Methylotrophicum, Eubacterium limosum, Clostridium aceticum, and combinations thereof); and an H₂-utilizing microorganism (such as Eubacterium aggregans, Blautia producta and combinations thereof) and combinations thereof. According to some such embodiments, at least one of the CO₂-utilizing microorganism; the CO-utilizing microorganism; and the H₂-utilizing organism is an acetogen.

According to an embodiment, the mixture of at least two different species comprises at least one non-CO₂ utilizing microorganism.

According to an embodiment, the mixture of at least two different species comprises at least one acetate forming microorganism and at least one acetate-utilizing microorganism.

According to an embodiment, the mixture of at least two different species comprises at least one lactate forming microorganism and at least one lactate-utilizing organism.

According to an aspect of some embodiments of the present invention, there is provided a pharmaceutical preparation comprising the composition as disclosed herein for use in the treatment of bloating.

According to an embodiment, the pharmaceutical preparation comprises between 10² and 10¹⁰ colony forming units of the microorganism, such as, for example, about 10², 10³, 10⁴, 10⁵, 10⁶, 10⁷, 10⁸, 10⁹, or 10¹⁰ colony forming units.

According to an embodiment, the pharmaceutical preparation comprises at least 10¹ Colony-Forming Units per milliliter of the microorganism. According to an embodiment, the pharmaceutical preparation comprises at least 10¹, at least 10², at least 10³, at least 10⁴, at least 10⁵, at least 10⁶, at least 10⁷, at least 10⁸, at least 10⁹ CFU per millimeter of the microorganism. According to an embodiment, the pharmaceutical preparation comprises up to 10¹⁰ CFU per millimeter of the microorganism.

According to an embodiment, the pharmaceutical preparation further comprises at least one selected from the group consisting of water, macronutrients, prebiotics, and probiotics and combinations thereof.

According to an embodiment, the macronutrients are selected from the group consisting of carbohydrates (such as glycogen, starch, cellulose, cellobiose, sucrose, maltose, lactose, mannose, galactose, fructose, inlulin, fructooligosaccharides, Galactooligosaccharide, mannan oligosaccharide, ribose, Trehalose, dextrose, multidextrin, pectin and combinations thereof); fats (such as saturated fat, unsaturated fat, monounsaturated fat, polyunsaturated fat, trans fat, triglycerides, cholesterol, fatty acids, and combinations thereof); proteins (such as free amino acids, peptides, and combinations thereof); and combinations thereof.

According to an aspect of some embodiments of the present invention, there is provided a method of treating bloating in an animal comprising administering to the animal an effective dose of a composition as disclosed herein, thereby improving the health of the animal.

According to an aspect of some embodiments of the present invention, there is provided a probiotic composition, comprising at least one genus of mixotrophic microorganism and a carrier for use in the treatment of bloating in an animal.

According to an aspect of some embodiments of the present invention, there is provided the use of a probiotic composition, comprising at least one genus of mixotrophic microorganism and a carrier in the manufacture of a medicament for use in the treatment of bloating in an animal.

According to an embodiment, the animal is selected from the group consisting of human, dog, cat, sheep, horse, chicken, swine, cattle, reptiles, goat, duck, turkey, fish, shrimp and crabs.

According to an embodiment, the microorganism colonizes at least a portion of a section of the digestive tract of the animal. According to some such embodiments, the section of the digestive tract is selected from the group consisting of duodenum, jejunum, ileum, small intestine, cecum and colon.

According to an embodiment, the microorganism colonizes at least 0.01%, at least 0.05%, at least 0.01%, at least 0.05%, at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80% or at least 90% of a section of the digestive tract of the animal.

According to an embodiment, the microorganism colonizes two or more sections of the digestive tract, such as two sections, three sections, four sections, five or more sections.

According to an embodiment, the probiotic composition is to be administered at least twice, such as twice, three times, four times, five times or more than five times. In some such embodiments, the probiotic composition is given at least twice within a period of at least one day, at least one week, at least two weeks or at least one month.

According to an embodiment, the probiotic composition is for administration before consumption of food, such as about one minute, about 5 minutes, about 10 minutes, about 15 minutes, about 20 minutes, about 30 minutes or even about one hour before beginning consumption of food.

According to an embodiment, the probiotic composition is for administration after consumption of food, such as within about one minute, within about 5 minutes, within about 10 minutes, within about 15 minutes, within about 20 minutes, within about 30 minutes or even within about one hour after ending consumption of food.

According to an embodiment, the probiotic is for administration together with food. According to some such embodiments, the probiotic composition is added to food consumed by an animal. According to some such embodiments, the probiotic is administered in a separate form while consuming food.

According to some embodiments, the probiotic composition is for coadministration together with an additional agent for the treating of bloating, such as one selected from the group consisting of antibiotics, prokinetics, antispasmodics, opioid agents, antidepressants, stimulants of fluid secretion and combinations thereof.

According to some embodiments, administering of the probiotic composition and the at least one additional agent are carried out independently, sequentially, simultaneously or concomitantly, or in a single composition, or any combination thereof.

According to an embodiment, the probiotic composition may be administered before administration of the at least one additional agent.

According to an embodiment, the probiotic composition may be administered after administration of the at least one additional agent.

According to an embodiment, the effective dose is administered once a day, twice a day, three times a day or more for a given period.

According to an embodiment, administering once a day comprises administering in the morning. According to an embodiment, administering once a day comprises administering in the afternoon. According to an embodiment, administering once a day comprises administering in the evening.

According to an embodiment, administering twice a day comprises administering in the morning and the afternoon. According to an embodiment, administering twice a day comprises administering in the morning and the evening. According to an embodiment, administering twice a day comprises administering in the afternoon and the evening.

According to an embodiment, administering three times a day comprises administering in the morning, afternoon and evening.

According to an embodiment, administering is carried out once, twice, three times or more per day for one day, two days, three days, four days, five days, six days, one week, two weeks, three weeks, one month, two months, three months, four months, five months, six months, seven months, eight months, nine months, ten months, eleven months, one year or more than one year.

According to an embodiment, administering is carried out by a route selected from the group consisting of enteral (such as oral, sublingual, buccal, gastric, duodenal or rectal) or parenteral (such as IV, intramuscular subcutaneous and transdermal routes).

According to an embodiment, administering is carried out by oral administration. According to some such embodiments, the composition is provided in the form of a solid oral dosage form, such as a tablet, capsule, sachet, powder, granule. According to some such embodiments, the composition is provided in the form of a liquid oral dosage form, such as a syrup, solution or dispersion. According to some embodiments, the composition further comprises one or more excipients, such as sweeteners, colorants, flavorants, stabilizers and the like.

According to an embodiment, the effective dose is configured to provide at least 10 organism Colony-Forming Units per gram wet feces of the animal on a second day after administering the composition.

According to an embodiment, the colonizing is configured to provide at least 100 Colony-Forming Units per gram wet feces of the animal on a twentieth day after administering the composition.

According to an embodiment, bloating is caused by a function or condition selected from the group consisting of an immune condition; a digestive condition; a pathogenic microorganism (including bacteria, viruses, fungi, protozoa); a disease of the gastrointestinal system (such as Irritable Bowel Syndrome (IBS), Celiac Disease or Inflammatory Bowel Disease, including Ulcerative Colitis, Crohn's Disease, Microscopic Colitis and Pauchitis or combinations thereof); an iatrogenic disturbance or dysbiosis caused by one selected from the group consisting of radiotherapy, chemotherapy, transplantation and combinations thereof; antibiotic-related diarrhea (such as related to treatment of Clostridium difficile-associated diarrhea or recurrent Clostridium difficile infections) and combinations thereof.

According to an embodiment, the effective dose is configured to provide formation of butyric acid at a rate sufficient to reach butyric acid concentration of at least 0.1 millimolar in the digestive track of the animal, such as at least 0.1, 0.2, 0.3, 04, 0.5, 0.6, 0.7, 0.8 or 0.9 millimolar. According to an embodiment, the effective dose is configured to provide formation of butyric acid at a rate sufficient to reach butyric acid concentration of no more than 1.0 millimolar in the digestive track of the animal.

According to an embodiment, the effective dose is configured to provide formation of butyric acid at a rate of at least 0.01 millimolar per hour in the digestive tract of the animal, such as at least 0.01, 0.05, 0.1, 0.2, 0.3, 04, 0.5, 0.6, 0.7, 0.8 or 0.9 millimolar per hour. According to an embodiment, the effective dose is configured to provide formation of butyric acid at a rate of no more than 1.0 millimolar per hour in the digestive tract of the animal.

### EXAMPLES

### Example 1

A probiotic composition is prepared comprising Eubacterium aggregans at a density of 10⁸ Colony Forming Units (CFU) per mL. The composition is prepared by growing E. aggregans in a nutritive growth media and harvesting the cells in mid-log growth. The cells are concentrated to 10⁸ CFU/mL in a sodium chloride solution containing 0.5 g/L cysteine-HCl. The composition is administered to a human subject suffering from bloating. E. aggregans adheres and propagates within the gastrointestinal (GI) tract and relieves the bloating by fixing CO₂ and H₂ evolved from other bacteria in the GI tract. The Eubacterium aggregan also produces butyric acid within the GI tract. Adherence and propagation of E. aggregans is confirmed using species specific PCR primers and by plating wet feces from the subject on selective, nutritive agar plates.

### Example 2

A probiotic composition is prepared comprising Clostridium carboxidivorans at a density of 10⁶ CFU/mL. The composition is prepared by growing C. carboxidivorans in a nutritive growth media until a sporulated population develops. The culture is treated with chloroform (at least 3% [v/v]) to kill all non-sporulated cells, and the spores are concentrated to 10⁶ CFU/mL in a sodium chloride solution containing 0.5 g/L cysteine-HCl. The composition is administered to a human subject suffering from bloating. C. carboxidivorans adheres and propagates within the gastrointestinal (GI) tract and relieves the bloating by fixing CO₂ and H₂ evolved from other bacteria in the GI tract. The Clostridium carboxidivorans also produces butyric acid within the GI tract. Adherence and propagation of C. carboxidivorans is confirmed using species specific PCR primers and by plating wet feces from the subject on selective, nutritive agar plates.

### Example 3

A probiotic composition is prepared comprising Eubacterium aggregans and Lactobacillus plantarum at a total density of 10⁸ Colony Forming Units (CFU) per mL. The composition is prepared by growing E. aggregans and L. plantarum in separate fermentations in a nutritive growth media and harvesting the cultures in mid-log growth. The cells are concentrated in a sodium chloride solution containing 0.5 g/L cysteine-HCl and mixed to achieve a total concentration of 10⁸ CFU/mL. The composition is administered to a human subject suffering from bloating. E. aggregans and L. plantarum adhere and propagate within the gastrointestinal (GI) tract. E. aggregans relieves the bloating by fixing CO₂ and H₂ evolved from L. plantarum and other bacteria in the GI tract. The E. aggregans also produces butyric acid within the GI tract. Adherence and propagation of E. aggregans and L. plantarum is confirmed using species specific PCR primers and by plating wet feces from the subject on selective, nutritive agar plates.

### Example 4

A probiotic composition is prepared comprising Clostridium carboxidivorans and C. butyricum at a total density of 10⁶ CFU/mL. The composition is prepared by growing C. carboxidivorans and C. butyricum in separate fermentations in a nutritive growth media until a sporulated population develops. The cultures are treated with chloroform (at least 3% [v/v]) to kill all non-sporulated cells. The spores are concentrated in a sodium chloride solution containing 0.5 g/L cysteine-HCl and mixed to achieve a total concentration of 10⁶ CFU/mL. The composition is administered to a human subject suffering from bloating. C. carboxidivorans and C. butyricum adhere and propagate within the gastrointestinal (GI) tract. C. carboxidivorans relieves the bloating by fixing CO₂ and H₂ evolved from C. butyricum and other bacteria in the GI tract. Both C. carboxidivorans and C. butyricum produce butyric acid within the GI tract. Adherence and propagation of the strains is confirmed using species specific PCR primers and by plating wet feces from the subject on selective, nutritive agar plates.

1. A method of treating bloating in a subject in need thereof, comprising administering to the subject an effective dose of a probiotic composition, comprising at least one genus of mixotrophic microorganism and a carrier.

2. (canceled)

3. The method of claim 1, wherein said carrier is configured to maintain an anaerobic environment for said microorganism.

4. The method of claim 1, wherein said microorganism is characterized by having at least one characteristic selected from the group consisting of butanoate metabolism, obligate anaerobic growth, gas fixation via the reductive acetyl-coenzyme A pathway, tolerance to bile salts at concentration greater than 0.05%, tolerance to pH of less than 4.5, spore formation, lactose utilization, and self-aggregation.

5. The method of claim 1, wherein said at least one is genus selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Enterococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

6-7. (canceled)

8. The method of claim 1, wherein said carrier is selected from the group consisting of oxygen reducer, cellulose, and carbohydrates.

9. The method of claim 1, said composition further comprising microorganisms of at least one species selected from the group consisting of Bacillus amyloliquefaciens; Bacillus timonensis; Bacillus coagulans; Bacillus licheniformis; Bacillus megaterium; Bacillus mesentricus; Bacillus polymyxa; Bacillus subtilis; Bifidobacterium animalis; Bifidobacterium bifidium; Bifidobacterium bifidus; Bifidobacterium thermophilus; Bifidobacterium longum; Bifidobacterium pseudolongum; Bifidobacterium lactis; Clostridium butyricum; Enterococcus faecium; Enterococcus faecalis; Escherichia coli; Lactobacillus thermophilus; Lactobacillus acidophilus; Lactobacillus brevis; Lactobacillus bulgaricus; Lactobacillus casei; Lactobacillus delbrueckii; Lactobacillus subspecies; Lactobacillus bulgaricus; Lactobacillus farciminis; Lactobacillus fermentum; Lactobacillus gallinarum; Lactobacillus jensenii; Lactobacillus paracasei; Lactobacillus plantarum; Lactobacillus reuteri; Lactobacillus rhamnosus; Lactobacillus lactis; Lactobacillus salivarius; Lactobacillus sobrius; Megasphaera elsdenii; Pediococcus acidolactici; Propionibacterium shermanii; Propionibacterium freudenreichii; Propionibacterium acidipropionici; Propionibacterium jensenii; Saccharomyces boulardii; Saccharomyces cerevisiae; Saccharomyces servisia; Streptococcus faecalis; Streptococcus faecium; Streptococcus gallolyticus; Streptococcus salivarius; Streptococcus subsp.; Streptococcus thermophilus; Streptococcus bovis and combinations thereof.

10. The method of claim 1, said composition comprising a mixture of at least two different species of mixotrophic microorganisms.

11-12. (canceled)

13. The method of claim 10, wherein said mixture of said at least two different species comprises at least one microorganism selected from the group consisting of a CO₂-utilizing microorganism; a CO-utilizing microorganism; and an H₂-utilizing microorganism.

14. The method of claim 13, wherein at least one of said CO₂-utilizing microorganism; said CO-utilizing microorganism; and said H₂-utilizing organism is an acetogen.

15. The method of claim 10, wherein said mixture of said at least two different species comprises at least one non-CO₂ utilizing microorganism.

16. The method of claim 10, wherein said mixture of said at least two different species comprises at least one acetate forming microorganism and at least one acetate-utilizing microorganism.

17. The method of claim 10, wherein said mixture of said at least two different species comprises at least one lactate forming microorganism and at least one lactate-utilizing organism.

18. (canceled)

19. The method of claim 1, said probiotic composition comprising between 10² and 10¹⁰ colony forming units of said microorganism.

20-22. (canceled)

23. The method of claim 1, wherein said subject is an animal selected from the group consisting of human, dog, cat, sheep, horse, chicken, swine, cattle, reptiles, goat, duck, turkey, fish, shrimp and crabs.

24. The method of claim 1, wherein said microorganism colonizes at least a portion of a section of the digestive tract of said animal.

25. The method of claim 24, wherein said section of the digestive tract is selected from the group consisting of duodenum, jejunum, ileum, small intestine, cecum and colon.

26-28. (canceled)

29. The method of claim 1, wherein said bloating is caused by a condition or function selected from the group consisting of an immune condition; a digestive condition; a pathogenic microorganism; a disease of the gastrointestinal system; an iatrogenic disturbance or dysbiosis caused by one selected from the group consisting of radiotherapy, chemotherapy, transplantation and combinations thereof; antibiotic-related diarrhea and combinations thereof.

30. The method of claim 29, wherein said disease of the gastrointestinal tract is selected from the group consisting of Irritable Bowel Syndrome (IBS), Celiac Disease and Inflammatory Bowel Disease.

31. The method of claim 30, wherein said Inflammatory Bowel Disease is selected from the group consisting of Ulcerative Colitis, Crohn's Disease, Microscopic Colitis and Pauchitis or combinations thereof.

32. The method of claim 29, wherein said antibiotic-related diarrhea is related to treatment of Clostridium difficile-associated diarrhea or recurrent Clostridium difficile infections.

33-35. (canceled)
